(19) United States
(12) Patent Application Publication
(10) Pub. No.: US 2004/0219125 A1
(43) Pub. Date: Nov. 4, 2004

(54) Oily thickened gel-like composition, emulsified composition using the composition and preparation method thereof

(76) Inventors: Tadashi Yoneda, Chiba (JP); Kazuo Furuya, Chiba (JP)

Correspondence Address: SUGHRUE MION, PLLC, 2100 PENNSYLVANIA AVENUE, N.W., SUITE 800, WASHINGTON, DC, 20037, US

(21) Appl. No.: 10/486,339

(22) PCT Filed: Aug. 9, 2002
(86) PCT No.: PCT/JP02/08180

(30) Foreign Application Priority Data
Aug. 10, 2001 (JP) 2001-244829
Oct. 5, 2001 (JP) 2001-309556

(51) Int. Cl. A61K 7/075; A61K 7/08

(52) U.S. Cl. 424/70.21

(57) ABSTRACT

An oily thickened gel-like which contains an anionic surfactant, water and/or a polyhydric alcohol and oily ingredient; an emulsified composition obtained by adding water to the oily thickened gel-like composition; a cosmetic composition containing the oily thickened gel-like composition or the emulsified composition. The anionic surfactant preferably has a lipopeptide structure. The oily thickened gel-like composition of the present invention is capable of containing an oily ingredient in a large amount and suitable for using as a cleansing cosmetic composition, a moisture retaining cosmetic composition and the like, extremely mild to cause little skin irritation.

### CROSS-REFERENCE TO THE RELATED APPLICATIONS

This is an application filed pursuant to 35 U.S.C. Section 111(a) with claiming the benefit of U.S. Provisional application Ser. No. 60/316,267 filed Sep. 4, 2001 and U.S. Provisional application Ser. No. 60/373,612 filed Apr. 19, 2002 under the provision of 35 U.S.C. Section 111(b), pursuant to 35 U.S.C. Section 119(e) (1).

### TECHNICAL FIELD

The present invention relates to an oily thickened gel-like composition, a preparation method of an emulsified composition using the oily thickened gel-like composition, the emulsified composition using the oily thickened gel-like composition and a cosmetic composition using these.

### BACKGROUND ART

For obtaining an oily gel-like composition, a method of blending silicic acid anhydride with a liquid oily ingredient, a method of blending a metal soap with a liquid oily ingredient, and a method of blending a polyhydric alcohol and a nonionic surfactant with a liquid oily ingredient have been heretofore known.

For example, JP-A-62-53910 (the term “JP-A” as used herein means an “unexamined published Japanese patent application”) (U.S. Pat. No. 4,767,625) discloses a liquid crystal-type cosmetic composition obtained from a hydrophilic nonionic surfactant, a water-soluble substance having a hydroxyl group within the molecule, an oily ingredient and water.

JP-A-3-141212 discloses a sol-state nonaqueous cosmetic composition obtained by blending a low polar oil, a metal soap and a nonionic surfactant. JP-A-6-48921 (U.S. Pat. No. 5,380,455) discloses a cleansing agent composition containing a fluorine-base polymer and a liquid oil. JP-A-9-255520 discloses a nonaqueous cosmetic composition containing from 40 to 70 mass % of a polyhydric alcohol, from 10 to 50 mass % of a non- to low-polar ingredient and from 1 to 30 mass % of a hydrophilic surfactant.

JP-A-10-139627 (U.S. Pat. No. 5,928,657) discloses a transparent gel containing a fatty phase, a carbohydrate fatty ester having from 5 to 7 carbon atoms and a polyhydric alcohol. JP-A-2000-26238 discloses a jelly cosmetic composition containing a diglycerol tetraoleate and an isoparaffin having from 16 to 30 carbon atoms. JP-A-2000-229816 discloses a non-solid, nonaqueous oily cosmetic composition containing a dextrin fatty acid ester, a heavy liquid paraffin and an oily ingredient which is liquid at an ordinary temperature. JP-A-2000-239123 discloses an oily gel-like composition comprising an unsaturated or branched alcohol having from 12 to 30 carbon atoms, an amphoteric surfactant and water.

However, the methods of blending a silicic acid anhydride, a metal soap, a nonionic surfactant or the like are deficient in that since these ingredients must be blended in a large amount so as to attain gelling, the extension on a skin is poor, skin irritation becomes a problem for some people, and due to the low oil content, the cleansing property is inferior to that of a liquid cleansing oil. Also, the method of blending a dextrin fatty acid ester has a problem in that the gel is easily crumbled and the stability is poor. As such, conventional oily thickened gel-like compositions all are insufficient.

On the other hand, an anionic surfactant is widely used as one of ingredients of cosmetics. Examples of the oily thickened gel-like composition using this include an oily ointment substrate disclosed in JP-A-61-257916, comprising from 5 to 10 mass % of a mixture of a hydrophilic polyglycerol higher fatty acid ester having an HLB (Hydrophilic Lipophilic Balance) of 9 or more and an anionic surfactant at a mass ratio of 100: (1 to 10), from 5 to 20 mass % of water and from 70 to 90 mass % of an oil phase ingredient.

### DISCLOSURE OF THE INVENTION

The object of the present invention is to provide an oily thickened gel-like composition extremely low in the skin irritation, capable of containing an oily ingredient in a large amount and suitable for a cleansing cosmetic composition, a moisture retaining cosmetic composition and the like; a preparation method of an emulsified composition using the oily thickened gel-like composition; the emulsified composition using the oily thickened gel-like composition; and a cosmetic composition comprising the oily thickened gel-like composition or the emulsified composition.

As a result of extensive investigations to attain the above-described object, the present inventors have found that an oily thickened gel-like composition comprising an anionic surfactant, water and/or a polyhydric alcohol, and an oily ingredient can be prepared. In addition, the present inventors have found that when this oily thickened gel-like composition is diluted with water, a stable emulsified composition can be obtained. The present invention has been accomplished based on these findings.

More specifically, the present invention relates to the following matters.

[1] An oily thickened gel-like composition comprising an anionic surfactant, water and/or a polyhydric alcohol, and an oily ingredient.

[2] The oily thickened gel-like composition as described in [1] above, wherein the anionic surfactant has a lipopeptide structure.

[3] The oily thickened gel-like composition as described in [1] above, wherein the anionic surfactant is contained in an amount of 0.01 to 45 mass % and water and/or the polyhydric alcohol is contained in an amount of 0.01 to 43 mass %.

[4] The oily thickened gel-like composition as described in [1] above, wherein the oily ingredient is contained in an amount of 1 to 99.9 mass %.

[5] The oily thickened gel-like composition as described in [2] above, wherein the anionic surfactant having a lipopeptide structure is a salt of surfactin and/or a salt of the homologue of surfactin.

[6] The oily thickened gel-like composition as described in [5] above, wherein the surfactin is a compound represented by the formula (I) or a composition comprising two or more kinds of the compound represented by the formula (I):

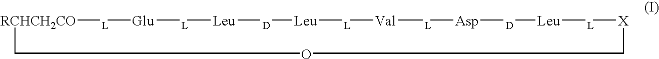

wherein X represents an amino-acid residue selected from the group consisting of leucine, isoleucine, valine, glycine, serine, alanine, threonine, asparagine, glutamine, aspartic acid, glutamic acid, lysine, arginine, cysteine, methionine, phenylalanine, thyrosin, tryptophan, histidine, proline, 4-hydroxyproline and homoserine, and R represents a normal alkyl group having from 8 to 14 carbon atoms, isoalkyl group having from 8 to 14 carbon atoms or anteisoalkyl group having from 8 to 14 carbon atoms.

[7] The oily thickened gel-like composition as described in [6] above, wherein X in formula (I) is an amino-acid residue selected from leucine, isoleucine or valine.

[8] The oily thickened gel-like composition as described in [5] above, wherein the salt is one or more salts selected from the group consisting of sodium salt, potassium salt, monoethanolamine salt, diethanolamine salt, triethanolamine salt, arginine salt and lysine salt.

[9] The oily thickened gel-like composition as described in [5] above, wherein the salt of surfactin and/or the salt of the homologue of the is sodium surfactin.

[10] The oily thickened gel-like composition as described in [1] above, wherein the polyhydric alcohol has three or more hydroxyl groups.

[11] The oily thickened gel-like composition as described in [10] above, wherein the polyhydric alcohol having three or more hydroxyl groups is one or more alcohols selected from the group consisting of glycerol, diglycerol, polyglycerol, sorbitol, multitol, erythritol, pentaerythritol, glucose, saccharose and trehalose.

[12] The oily thickened gel-like composition as described in [1] above, wherein the oily ingredient is a liquid oil.

[13] The oily thickened gel-like composition as described in [12] above, wherein the liquid oil is one or more liquid oils selected from the group consisting of mineral oil, vegetable oil, hydrocarbon, ester oil, silicone oil and triglyceride.

[14] The oily thickened gel-like composition as described in [12] above, wherein the liquid oils one or more liquid oils selected from the group consisting of glycerol tri(2-ethylhexanoate), cetyl 2-ethylhexanoate, 2-octyldodecyl myristate, isopropyl myristate, liquid paraffin, squalane, jojoba oil, olive oil, rice embryo oil, sunflower seed oil and clove oil.

[15] The oily thickened gel-like composition as described in [13] above, wherein the silicone oil is one or more liquid oils selected from the group consisting of dimethyl silicone oil such as octamethyltrisiloxane, decamethyltetrasiloxane, methyl-polysiloxane and high-polymerized methylpolysiloxane, cyclic silicone oil such as octamethylcyclotetrasiloxane, decamethylcyclopentasiloxane, dodecamethylcyclohexasiloxane, methylcyclopolysiloxane and methylpolycyclosiloxane, poly ether modified silicone oil, methylphenyl silicone oil such as methylphenylpolysiloxane.

[16] The oily thickened gel-like composition as described in [12] above, wherein the liquid oil comprises a liquid oil having a property of ultraviolet absorption.

[17] The oily thickened gel-like composition as described in [16] above, wherein the liquid oil having a property of ultraviolet absorption is 2-ethylhexyl p-methoxycinnamate or 2-ethylhexyl p-methoxycinnamate solution of 4-t-butyl-4′ methoxybenzoylmethane.

[18] The oily thickened gel-like composition as described in [1] above, which is transparent.

[19] The oily thickened gel-like composition as described in [18] above, wherein a difference of refractive index n_{D}²⁰ between oil phase and aqueous phase in the oily thickened gel-like composition is 0.01 or less.

[20] A method for preparing an emulsified composition, comprising adding water to the oily thickened gel-like composition described in any one of [1] to [19] above to emulsify the composition.

[21] An emulsified composition obtained by the preparation method described in [20] above.

[22] A cosmetic composition containing the emulsified composition described in [21] above.

[23] A cosmetic composition containing the oily thickened gel-like composition described in any one of [1] to [19] above.

### DETAILED DESCRIPTION OF THE INVENTION

The present invention is described in detail below.

The anionic surfactant for use in the present invention may be any anionic surfactant insofar as an oily thickened gel-like composition can be prepared, but is preferably an anionic surfactant having a lipopeptide structure. Examples of the anionic surfactant include a salt of surfactin and/or a salt of the homologue of surfactin.

Surfactin indicates a compound represented by formula (I), or a composition comprising two or more kinds of the compound.

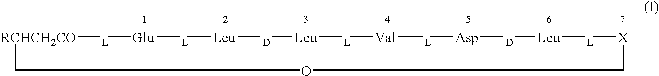

In the formula (I), X represents an amino-acid residue selected from the group consisting of leucine, isoleucine, valine, glycine, serine, alanine, threonine, asparagine, glutamine, aspartic acid, glutamic acid, lysine, arginine, cysteine, methionine, phenylalanine, thyrosin, tryptophan, histidine, proline, 4-hydroxyproline and homoserine. Leucine, isoleucine and valine are preferable as X.

R is a normal alkyl group having from 8 to 14 carbon atoms, isoalkyl group having from 8 to 14 carbon atoms or anteisoalkyl group having from 8 to 14 carbon atoms. The normal alkyl group is a straight chain alkyl group. The isoalkyl group usually has a structure of (CH₃)₂CH—(CH₂)ₙ—. The anteisoalkyl group usually has a structure of CH₃—CH₂—CH(CH₃)—(CH₂)ₙ—.

The homologue of surfactin indicates a compound represented by the formula (I) wherein one or more amino acids are replaced with other kind of amino acids. Specifically, the second L-leucine, the forth L-valine, the sixth D-leucine and the like in the formula (I) are replaced with other amino acids, but it is not limited to these. Hereinbelow, a surfactin or a homologue thereof are sometimes referred to simply as surfactin.

The surfactin is usually produced by a prokaryote. The prokaryote used in general is a microorganism of the genus Bacillus, such as Bacillus subtilis IAM1213 strain, IAM1069 strain, IAM1259 strain, IAM1260 strain, IF03035 strain and ATCC 21332 strain.

The surfactin can be easily obtained by culturing and purifying this microorganism. In the purification, for example, the culture solution is rendered acidic by adding hydrochloric acid or the like and the precipitated surfactin is separated by filtration, dissolved in an organic solvent such as methanol, and then appropriately subjected to ultrafiltration, treatment with activated carbon, crystallization or the like. In place of the precipitation by the addition of an acid, precipitation by the addition of a calcium salt may also be used (Biochem. Bioph. Res. Commun., 31: 488-494 (1968)).

As described above, besides the ones produced by a prokaryote such as microorganism of the genus Bacillus, surfactins obtained by other production processes, for example, chemical synthesis method, can be employed in the present invention.

As seen from the formula (I), the surfactin can be used as an inorganic salt or an organic salt. The metal as a counter ion is not limited on the kind insofar as it forms a salt with surfactin, and an alkali metal such as sodium, potassium or lithium, or an alkaline earth metal such as calcium or magnesium may be used.

Examples of the organic salt include trimethylamine, triethylamine, tributylamine, monoethanolamine, diethanolamine, triethanolamine, lysine, arginine and choline.

Among these, preferred are sodium, potassium, monoethanolamine, diethanolamine, triethanolamine, lysine and arginine, and more preferred is sodium.

As for the sodium salt of surfactin, sodium surfactin commercially available from Showa Denko K.K. under the trade name of Aminofect (registered trademark of Showa Denko K.K.) can be used.

The anionic surfactant having a lipopeptide structure, such as a salt of surfactin and a salt of the homologue of surfactin, is extremely low in the irritation to skin. The reason why the salt of surfactin and the salt of the homologue of surfactin is low in the irritation to skin is considered because surfactin or the homologue thereof is a complicated ring compound and bulky and therefore, the percutaneous permeability thereof is low. Also, since the salt of surfactin and the salt of the homologue of surfactin have a masking effect of surrounding a skin irritating substance, the irritation of the irritating substance is considered to decrease. According to the present invention, an oily thickened gel-like composition having extremely low irritation to the skin can be provided.

The content of the anionic surfactant in the oily thickened gel-like composition of the present invention is not particularly limited, but is preferably from 0.01 to 45 mass %, more preferably from 0.1 to 20 mass %. If the content of sodium surfactin is less than 0.01 mass %, gelling does not proceed sufficiently, whereas if it exceeds 45 mass %, the viscosity is excessively increased and this is not preferred.

The oily thickened gel-like composition of the present invention comprises the anionic surfactant, water and/or the polyhydric alcohol, and the oily ingredient as described above.

As for the polyhydric alcohol for use in the present invention, a polyhydric alcohol commonly used in cosmetic compositions can be used without any particular limitation insofar as the oily thickened gel-like composition of the present invention can be prepared.

Examples of this polyhydric alcohol include propylene glycol, dipropylene glycol, polypropylene glycol, glycerol, diglycerol, polyglycerol, ethylene glycol, diethylene glycol, polyethylene glycol, 1,3-butanediol, sorbitol, multitol, erythritol, pentaerythritol, glucose, saccharose, maltose, xylose and trehalose. These may be used individually or in combination of two or more thereof.

Among these, preferred are the polyhydric alcohol having three or more hydroxyl groups such as glycerol, diglycerol, polyglycerol, sorbitol, multitol, erythritol, pentaerythritol, glucose, saccharose, maltose, xylose, trehalose and the like, and more preferred are glycerol and sorbitol.

The content of polyhydric alcohol in the oily thickened gel-like composition of the present invention is preferably from 0.01 to 43 mass %, more preferably from 0.05 to 39 mass % and even more preferably from 0.1 to 36 mass %.

When the oily thickened gel-like composition of the present invention contains water, the gel having low viscosity can be prepared, and when the oily thickened gel-like composition is used as gel substance in cosmetic compound, it improves a feeling of the use. Moreover, the gel whose appearance is transparent can be prepared by adjusting the refractive indices of an oil phase and an aqueous phase to the same level. Thus-obtained transparent gel can be improved in a design property.

As for the oily ingredient for use in the present invention, an oily ingredient commonly used in cosmetic compositions can be used without any particular limitation, however, an oily ingredient which is liquid or paste at 25° C. and 1 atm is preferred and liquid oil is more preferable.

Examples of this liquid oil include hydrocarbons, higher alcohol esters, higher fatty acid esters, triglycerides, silicone oils, higher alcohols, higher fatty acids, animal and vegetable oils, cholesterol fatty acid esters, sterols, sterol esters and polyphenols. Preferred examples thereof include meneral oil, liquid paraffin, squalane, isopropyl palmitate, isopropyl myristate, isooctyl myristate, isotridecyl myristate, octadecyl myristate, 2-octyldodecyl myristate, isostearyl cholesteryl ester, glycerol tri(2-ethylhexanoate), cetyl 2-ethylhexanoate, sunflower seed oil, olive oil, jojoba oil, tsubaki oil, grape seed oil, avocado oil, macadamia nut oil, almond oil, rice embryo oil, clove oil, orange oil, oil of bitter orange, dimethyl silicone oil such as octamethyltrisiloxane, decamethyltetrasiloxane, methylpolysiloxane, high-polymerized methylpolysiloxane and the like; cyclic silicone oil such as octamethylcyclotetrasiloxane, decamethylcyclopentasiloxane, dodecamethylcyclohexasiloxane, methylcyclopolysiloxane, methylpolycyclosiloxane and the like; poly ether modified silicone oil; methylphenyl silicone oil such as methylphenyl-polysiloxane and the like.

These may be used individually or in combination of two or more thereof.

The content of the oily ingredient in the oily thickened gel-like composition of the present invention is preferably from 1 to 99.9 mass %, more preferably from 50 to 95 mass %.

An ultraviolet absorbent can be blended in the oily thickened gel-like composition of the present invention. The ultraviolet absorbent which can be used in the present invention indicates a substance that is used for sun-screen cosmetics and cuts ultraviolet A wave, ultraviolet B wave or both thereof to reduce a harmfulness of ultraviolet for the skin.

Examples of the ultraviolet absorbent include p-aminobenzoic acid derivatives such as p-aminobenzoic acid, glyceryl-p-aminobenzoate, amyl-p-N,N-dimethylaminobenzoate, 2-ethylhexyl-p-N,N-dimethylaminobenzoate and the like; cinnamic acid derivatives such as 2,4-diisopropyl methylcinnamate, 2,4-diisopropyl ethylcinnamate, potassium p-methoxycinnamate, sodium p-methoxycinnamate, isopropyl p-methoxycinnamate, 2-ethylhexyl p-methoxycinnamate, 2-ethoxyethyl p-methoxy-cinnamate, ethyl p-ethoxycinnamate and the like; benzophenone derivatives such as 2,4-dihydroxybenzophenone, 2,2′,4,4′-tetrahydroxybenzophenone, sodium 2-hydroxy-4-methoxy-5-sulfobenzophenone, 2-hydroxy-4-methoxybenzophenone-5-sulfonic acid, 2-hydroxy-4-methoxybenzophenone, 2,2′-dihydroxy-4,4′-dimethoxybenzophenone, sodium 2,2′-dihydroxy-4,4′-dimethoxy-5-sulfobenzophenone and the like; salicylic acid derivatives such as 2-ethylhexyl salicylate, phenyl salicylate, 3,3,5-trimethylcyclohexylsalicylate and the like; 2-(2′-hydroxy-5′-methoxyphenyl)benzotriazole, 4-tert-butyl-4′-methoxybenzoyl-methane and the like.

Among these, the ones which are solid at ordinary temperature can be dissolved or dispersed in liquid oil upon use. The ones which are liquid or paste at ordinary temperature can be used as is for liquid oil ingredient, and can be used in a mixture with other liquid oils. Preferred examples of ultraviolet absorbents which can be used as is for liquid oil ingredient are 2-ethylhexyl p-methoxycinnamate or 2-ethylhexyl p-methoxy-cinnamate solution of 4-tert-butyl-4′-methoxybenzoylmethane.

An antioxidant and a perfume can also be blended in the oily thickened gel-like composition of the present invention. Among these, the ones which are solid at ordinary temperature can be dissolved or dispersed in liquid oil upon use. The ones which are liquid or paste at ordinary temperature can be used as is, and can be used in a mixture with other liquid oils. Examples of the antioxidant which can be used include, tocopherol, tocopherol acetate, vitamin As such as retinoic acid, retinoic acid ester, retinol and retinoid.

The oily thickened gel-like composition of the present invention can be prepared by dissolving anionic surfactant in polyhydric alcohol and/or water and adding oily ingredient thereto little by little while stirring. In the case where water is used with polyhydric alcohol, water may be added after the oily ingredient is added. The oily ingredient may be added by installment in a predetermined amount each (divided addition), and also it may be added continuously (continuous addition). In the case of divided addition, the oily ingredient is added at one time in an amount of preferably 60 mass % or less, more preferably 30 mass % or less, particularly preferably 10 mass % or less based on the amount of polyhydric alcohol and/or water which have already been blended, and then the mixture is made uniform by stirring. This step is repeated until the amount of the oily ingredient added reaches the required amount. In the case of continuous addition, the addition speed is preferably 60 mass % per minutes or less, more preferably 30 mass % per minutes or less, particularly preferably 10 mass % per minutes or less based on the amount of polyhydric alcohol and/or water which has already been blended.

Further, other ingredients may be added before adding the oily ingredient, or by dissolving or dispersing in the oily ingredient, or after adding the total amount of the oily ingredient, or in process of adding the oily ingredient. All the amount of polyhydric alcohol and/or water may be added first, or a part thereof maybe added first, then the rest can be added later.

By adding water to the oily thickened gel-like composition of the present invention, an emulsified composition having extremely excellent stability and being suitable for a cosmetic composition such as cream or lotion can be obtained. The preparation method of an emulsified composition using the oily thickened gel-like composition, and the emulsified composition obtained are also included in the present invention.

The appearance of the oily thickened gel-like composition in the present invention can be transparent by adjusting the composition of the oily thickened gel-like composition. By the term “transparent” used here, it is intended that letters printed on a newspaper can be identified through a transparent vial having a diameter of 30 mm with the oily thickened gel-like composition in it.

The difference in the refractive index n_{D}²⁰ measured according to JIS K0062 between oil phase and aqueous phase of the oily thickened gel-like composition is adjusted to 0.01 or less, preferably 0.005 or less to obtain such a transparent appearance.

The oily thickened gel-like composition in the present invention can be suitably used for a cosmetic composition, for example, a basic skin care cosmetic composition such as cream, lotion, cleansing gel and cleansing cream; a make up cosmetic composition such as foundation, eyeshadow, lipstick and lipgloss; a hair cosmetic composition such as hair cream, styling gel and hair wax; a cleansing agent such as shampoo, rinse, hand soap, body soap and facial wash foam. These cosmetic compositions are also included in the present invention.

In the case of using the oily thickened gel-like composition for a cosmetic composition, arbitrary ingredients commonly used for a cosmetic composition can be blended.

Examples of the ingredient include hydrocarbons such as vaseline and microcrystalline wax, esters such as octyldodecyl myristate and isopropyl myristate, triglycerides such as glyceryl triisooctanoate and olive oil, silicone oils such as methylphenylpolysiloxane and methylpolysiloxane, higher alcohols such as cetanol and behenyl alcohol, fatty acids such as stearic acid and oleic acid, polyhydric alcohols such as glycerol, 1,3-butanediol and propylene glycol, lower alcohols such as ethanol and isopropyl alcohol, nonionic surfactant, anionic surfactant, cationic surfactant, amphoteric surfactant, thickener, ultraviolet absorbent, antioxidant., emollient, emulsifier, solubilizing agent, anti-inflammatory, moisture retaining agent, antiseptic, microbicide, pH adjusting agent, dye, perfume, powders and water.

The cosmetic composition of the present invention may also contain an existing cosmetic ingredient in a general concentration. Examples of the cosmetic ingredient include those described in Japanese Standards of Cosmetic Ingredients (JSCI), 2nd Edition, Annotation, compiled by Nippon Koteisho Kyokai, issued by Yakuji Nippo, Ltd. (1984), Specifications of Ingredient Other Than Those Listed in JSCI, supervised by Examination Division, Pharmaceutical Affairs Bureau, Ministry of Health and Welfare, issued by Yakuji Nippo, Ltd. (1993), Specifications of Ingredient Other Than Those Listed in JSCI, Supplement, supervised by Examination Division, Pharmaceutical Affairs Bureau, Ministry of Health and Welfare, issued by Yakuji Nippo, Ltd. (1993), The Comprehensive Licensing Standards of Cosmetics by Category, supervised by Examination Division, Pharmaceutical Affairs Bureau, Ministry of Health and Welfare, issued by Yakuji Nippo, Ltd. (1993), and Kesho-hin Genryo Jiten (Handbook of Cosmetic Ingredients), Nikko Chemicals (1991). All cosmetic ingredients described in these publications can be used.

Among these arbitrary ingredients, preferred in the cosmetic composition of the present invention are nonionic surfactants, higher fatty acids and higher alcohols, more preferred are stearic acid and behenyl alcohol. The content thereof is preferably from 0.01 to 10 mass %, more preferably from 0.1 to 5 mass %.

The thus-obtained cosmetic composition of the present invention is free of skin irritation and is very excellent as a cleansing agent, a moisture retaining agent, a cream or a lotion.

### BEST MODE FOR CARRYING OUT THE INVENTION

The present invention is described in greater detail below by referring to Examples, however, the present invention is not limited to these Examples. In addition, the glycerol described below had purity of glycerol content of 98 mass % or more. As sodium surfactin, Aminofect (registered trademark: product of Showa Denko K.K.) was used. The values indicated by “%” are values in mass percentage.

### EXAMPLE 1

### Nonaqueous Oily Thickened Gel-Like Composition

1 part by mass of sodium surfactin was dissolved in 20 parts by mass of glycerol. 2 parts by mass of squalane was added thereto while stirring until becoming uniform. This operation was repeated five times. After that, 4 parts by mass of squalane was added thereto while stirring until becoming uniform. This operation was repeated five times. Subsequently, 6 parts by mass of squalane was added thereto while stirring until becoming uniform. This operation was repeated three times. Then, 8 parts by mass of squalane was added thereto while stirring until becoming uniform. This operation was repeated four times to obtain nonaqueous oily thickened gel-like composition. The composition amount of each ingredient is as follows.

| | |
|---|---|
| Squalane | 80 parts by mass |
| Glycerol | 20 parts by mass |
| Sodium surfactin | 1 part by mass |

The obtained nonaqueous oily thickened gel-like composition had a translucent appearance and when a cleansing cosmetic composition was prepared using this, unlike squalane, liquid dripping did not occur and an excellent cleansing property was exhibited. When a moisture retaining cosmetic composition was prepared using this, good attachment to skin and usability for feeling the smooth skin were provided without skin irritation.

### EXAMPLE 2

### Transparent Oily Thickened Gel-Like Composition

In accordance with the following preparation, sodium surfactin and water were dissolved in glycerol and thereto, squalane in small portions each was added while stirring.

| | |
|---|---|
| Squalane | 77% |
| Glycerol | 19% |
| Water | 3% |
| Sodium surfactin | 1% |

The obtained oily thickened gel-like composition had a transparent appearance and when a cleansing cosmetic composition was prepared using this, unlike squalane, liquid dripping did not occur and an excellent cleansing property was exhibited. When a moisture retaining cosmetic composition was prepared using this, good attachment to skin and usability for feeling the smooth skin were provided without skin irritation.

### EXAMPLE 3

### Transparent Oily Thickened Gel-Like Composition

In accordance with the following preparation, sodium surfactin was dissolved in glycerol and thereto, squalane in small portions each was added while stirring. Additionally, water and 1,3-butandiol was slowly added thereto while stirring.

| | |
|---|---|
| Squalane | 60% |
| Glycerol | 23% |
| 1,3-butandiol | 13% |
| Water | 3% |
| Sodium surfactin | 1% |

The obtained oily thickened gel-like composition had a transparent appearance, low viscosity and excellent usability. When a cleansing cosmetic composition was prepared using this, unlike squalane, liquid dripping did not occur and an excellent cleansing property was exhibited. When a moisture retaining cosmetic composition was prepared using this, good attachment to skin and usability for feeling the smooth skin were provided without skin irritation.

### EXAMPLE 4

### Nonaqueous Oily Thickened Gel-Like Composition

In accordance with the following preparation, sodium surfactin was dissolved in glycerol and thereto, jojoba oil in small portions each was added while stirring.

| | |
|---|---|
| Jojoba oil | 79% |
| Glycerol | 20% |
| Sodium surfactin | 1% |

The obtained nonaqueous oily thickened gel-like composition had a transparent appearance and when a cleansing cosmetic composition was prepared using this, unlike jojoba oil, liquid dripping did not occur and an excellent cleansing property was exhibited. When a moisture retaining cosmetic composition was prepared using this, good attachment to skin and usability for feeling the refreshed skin were provided without skin irritation.

### EXAMPLE 5

### Nonaqueous Oily Thickened Gel-Like Composition

In accordance with the following preparation, sodium surfactin was dissolved in glycerol and thereto, olive oil in small portions each was added while stirring.

| | |
|---|---|
| Olive oil | 79% |
| Glycerol | 20% |
| Sodium surfactin | 1% |

The obtained nonaqueous oily thickened gel-like composition had a transparent appearance and when a cleansing cosmetic composition was prepared using this, unlike olive oil, liquid dripping did not occur and an excellent cleansing property was exhibited. When a moisture retaining cosmetic composition is prepared using this, good attachment to skin and usability for feeling the smooth skin were provided without skin irritation.

### EXAMPLE 6

### Nonaqueous Oily Thickened Gel-Like Composition

In accordance with the following preparation, sodium surfactin was dissolved in glycerol and thereto, mineral oil in small portions each was added while stirring.

| | |
|---|---|
| Mineral oil | 79% |
| Glycerol | 20% |
| Sodium surfactin | 1% |

The obtained nonaqueous oily thickened gel-like composition had a transparent appearance and when a cleansing cosmetic composition was prepared using this, unlike the cleansing oil of Comparative Example 3, liquid dripping did not occur and an excellent cleansing property was exhibited.

### EXAMPLE 7

### Nonaqueous Oily Thickened Gel-Like Composition

In accordance with the following preparation, sodium surfactin was dissolved in glycerol and thereto, liquid paraffin in small portions each was added while stirring.

| | |
|---|---|
| Liquid paraffin | 79% |
| Glycerol | 20% |
| Sodium surfactin | 1% |

Production Method:

The obtained nonaqueous oily thickened gel-like composition had a transparent appearance and when a cleansing cosmetic composition was prepared using this, unlike the cleansing oil of Comparative Example 3, liquid dripping did not occur and an excellent cleansing property was exhibited.

### EXAMPLE 8

### Nonaqueous Oily Thickened Gel-Like Composition

In accordance with the following preparation, sodium surfactin was dissolved in glycerol and thereto, methylpolysiloxane in small portions each was added while stirring.

| | |
|---|---|
| Methylpolysiloxane | 79% |
| Glycerol | 20% |
| Sodium surfactin | 1% |

The obtained nonaqueous oily thickened gel-like composition had a milky appearance and can be used as gel cosmetic composition for skin, hair and the like. When this was blended as the substrate for a cream or a lotion, an extremely fine and stable emulsion was obtained.

### EXAMPLE 9

### Transparent Oily Thickened Gel-Like Composition

In accordance with the following preparation, sodium surfactin was dissolved in glycerol and thereto, methylpolysiloxane in small portions each was added while stirring. Additionally, water was slowly added thereto while stirring.

| | |
|---|---|
| Methylpolysiloxane | 73% |
| Glycerol | 13% |
| Water | 13% |
| Sodium surfactin | 1% |

The obtained oily thickened gel-like composition had a transparent appearance and can be used as a gel cosmetic composition for skin, hair and the like. When this was blended as the substrate for a cream or a lotion, an extremely fine and stable emulsion was obtained.

### EXAMPLE 10

### Transparent Oily Thickened Gel-Like Composition

In accordance with the following preparation, sodium surfactin was dissolved in glycerol and thereto, methylpolysiloxane in small portions each was added while stirring. Additionally, water was slowly added thereto while stirring.

| | |
|---|---|
| Methylpolysiloxane | 67% |
| Glycerol | 16% |
| Water | 16% |
| Sodium surfactin | 1% |

The obtained oily thickened gel-like composition had a transparent appearance and a slight fluidity and can be used as gel cosmetic composition for skin, hair and the like. When this was blended as the substrate for a cream or a lotion, an extremely fine and stable emulsion was obtained.

### EXAMPLE 11

### Transparent Oily Thickened Gel-Like Composition

In accordance with the following preparation, sodium surfactin was dissolved in glycerol and thereto, methylpolysiloxane in small portions each was added while stirring. Additionally, water was slowly added thereto while stirring.

| | |
|---|---|
| Methylpolysiloxane | 57% |
| Glycerol | 21% |
| Water | 21% |
| Sodium surfactin | 1% |

The obtained oily thickened gel-like composition had a transparent appearance and a fluidity and can be used as gel cosmetic composition for skin, hair and the like. When this was blended as the substrate for a cream or a lotion, an extremely fine and stable emulsion was obtained.

### EXAMPLE 12

### Emulsion

In accordance with the following preparation, sodium surfactin was dissolved in glycerol and thereto, methylpolysiloxane in small portions each was added while stirring. Additionally, water was slowly added thereto while stirring.

| | |
|---|---|
| Methylpolysiloxane | 57% |
| Glycerol | 15% |
| Water | 27% |
| Sodium surfactin | 1% |

The obtained emulsion had a milky appearance and was an extremely fine and stable emulsion. When this was blended as the substrate for a lotion and the like and usability for feeling the smooth skin were provided without skin irritation.

### EXAMPLE 13

### Nonaqueous Oily Thickened Gel-Like Composition

In accordance with the following preparation, sodium surfactin was dissolved in glycerol and thereto, rice embryo oil in small portions each was added while stirring.

| | |
|---|---|
| Rice embryo oil | 79% |
| Glycerol | 20% |
| Sodium surfactin | 1% |

This nonaqueous oily thickened gel-like composition had a transparent appearance and when a moisture retaining cosmetic composition was prepared using this, unlike rice embryo oil, liquid dripping did not occur and good attachment to skin, usability for feeling the soft skin were provided without skin irritation.

### EXAMPLE 14

### Nonaqueous Oily Thickened Gel-Like Composition

In accordance with the following preparation, sodium surfactin was dissolved in glycerol and thereto, squalane in small portions each was added while stirring.

| | |
|---|---|
| Squalane | 94% |
| Glycerol | 5% |
| Sodium surfactin | 1% |

The obtained nonaqueous oily thickened gel-like composition had a transparent appearance and when a cleansing cosmetic composition was prepared using this, unlike squalane, liquid dripping did not occur and an excellent cleansing property was exhibited. When a moisture retaining cosmetic composition was prepared using this, good attachment to skin and usability for feeling the smooth skin were provided without skin irritation.

### EXAMPLE 15

### Nonaqueous Oily Thickened Gel-Like Composition

In accordance with the following preparation, sodium surfactin was dissolved in glycerol and thereto, olive oil in small portions each was added while stirring.

| | |
|---|---|
| Olive oil | 92% |
| Glycerol | 7% |
| Sodium surfactin | 1% |

The obtained nonaqueous oily thickened gel-like composition had a transparent appearance and when a cleansing cosmetic composition was prepared using this, unlike olive oil, liquid dripping did not occur and an excellent cleansing property was exhibited. When a moisture retaining cosmetic composition was prepared using this, swift attachment to skin, good extension and usability for feeling the smooth skin were provided without skin irritation.

### EXAMPLE 16

### Nonaqueous Oily Thickened Gel-Like Composition

In accordance with the following preparation, sodium surfactin was dissolved in 1,3-butanediol and thereto, squalane in small portions each was added while stirring.

| | |
|---|---|
| Squalane | 67% |
| 1,3-Butanediol | 32% |
| Sodium surfactin | 1% |

The obtained nonaqueous oily thickened gel-like composition had a transparent appearance and when a cleansing cosmetic composition was prepared using this, unlike squalane, liquid dripping did not occur and an excellent cleansing property was exhibited. When a moisture retaining cosmetic composition was prepared using this, swift attachment to skin, good extension and usability for feeling the smooth skin were provided without skin irritation.

### EXAMPLE 17

### Oily Thickened Gel-Like Composition

In accordance with the following preparation, sodium surfactin was dissolved in 70% aqueous sorbitol solution and thereto, squalane in small portions each was added while stirring.

| | |
|---|---|
| Squalane | 79% |
| 70% aqueous sorbitol solution | 20% |
| Sodium surfactin | 1% |

The obtained oily thickened gel-like composition had a translucent appearance and when a cleansing cosmetic composition was prepared using this, unlike squalane, liquid dripping did not occur and an excellent cleansing property was exhibited. When a moisture retaining cosmetic composition was prepared using this, swift attachment to skin, good extension and usability for feeling the smooth skin were provided without skin irritation.

### EXAMPLE 18

### Oily Thickened Gel-Like Composition

In accordance with the following preparation, sodium surfactin was dissolved in water and thereto, squalane in small portions each was added while stirring.

| | |
|---|---|
| Squalane | 73% |
| Water | 18% |
| Sodium surfactin | 9% |

The obtained oily thickened gel-like composition had a translucent appearance and when a cleansing cosmetic composition was prepared using this, unlike squalane, liquid dripping did not occur and an excellent cleansing property was exhibited. When a moisture retaining cosmetic composition was prepared using this, swift attachment to skin and usability for feeling the smooth skin were provided without skin irritation.

### EXAMPLE 19

### Emollient Cream

In accordance with the following preparation A, sodium surfactin was dissolved in glycerol and thereto, an oily ingredient in small portions each was added while stirring to prepare a gel. The gel of preparation A was heated to 70° C., liquid of preparation B which was heated to 70° C. to dissolve was added thereto and the mixture was made uniform by stirring with a homo-mixer at 5000 rpm for 5 minutes. Water was added thereto in amount of following preparation C while further stirring. The resulting mixture was cooled to 40° C. or less while paddle-stirring, and was left standing.

preparation A:

| | |
|---|---|
| Squalane | 12% |
| Jojoba oil | 4% |
| Methylpolysiloxane | 0.2% |
| Glycerol | 4% |
| Sodium surfactin | 1% |

preparation B:

| | |
|---|---|
| Stearic acid | 4% |
| Behenyl alcohol | 2% |
| Cetanol | 2% |
| Paraffin | 3% |
| Triglycerol 2-ethylhexanoate | 8% |
| Antiseptic | opt. |

preparation C:

| | |
|---|---|
| Water | bal. |

The obtained cream had a milky appearance and an excellent emollient property, and provided usability for feeling the smooth skin without skin irritation.

### EXAMPLE 20

### Milky Lotion

In accordance with the following preparation A, sodium surfactin was dissolved in glycerol and thereto, squalane in small portions each was added while stirring to prepare a gel. The gel of preparation A was heated to 70° C., liquid of preparation B which was heated to 70° C. to dissolve was added thereto and the mixture was made uniform by stirring with a homo-mixer at 5000 rpm for 5 minutes. Water was added thereto in amount of following preparation C while further stirring. The resulting mixture was cooled to 40° C. or less while paddle-stirring, and was left standing.

preparation A:

| | |
|---|---|
| Squalane | 16% |
| Glycerol | 4% |
| Sodium surfactin | 1% |

preparation B:

| | |
|---|---|
| Stearic acid | 0.5% |
| Behenyl alcohol | 2% |
| Antiseptic | opt. |

preparation C:

| | |
|---|---|
| Arginine | 0.1% |
| Water | bal. |

The obtained milky lotion was an extremely fine and stable emulsion having a milky appearance and an excellent moisture retaining property and provided usability for feeling the smooth skin without skin irritation.

### EXAMPLE 21

### Lotion

In accordance with the following preparation A, sodium surfactin was dissolved in glycerol and thereto, an oily ingredient in small portions each was added while stirring to prepare a gel. The gel preparation A was heated to 80° C., liquid of preparation B which was heated to 80° C. to dissolve was added thereto and the mixture was made uniform by stirring with a homo-mixer at 5000 rpm for 5 minutes. Aqueous solution of arginine as following preparation C was added thereto while further stirring. The resulting mixture was cooled to 40° C. or less while paddle-stirring, and was left standing.

preparation A:

| | |
|---|---|
| Squalane | 16% |
| Glycerol | 4% |
| Sodium surfactin | 0.2% |

preparation B:

| | |
|---|---|
| Stearic acid | 0.5% |
| Behenyl alcohol | 2% |

preparation C:

| | |
|---|---|
| L-Arginine | 0.1% |
| Water | bal. |

The obtained lotion provided an excellent moisture retaining property and usability for feeling the smooth skin without skin irritation.

### EXAMPLE 22

### Cream

In accordance with the following preparation A, sodium surfactin was dissolved in glycerol and thereto, an oily ingredient in small portions each was added while stirring to prepare a gel. The gel preparation A was heated to 80° C., liquid of preparation B which was heated to 80° C. to dissolve was added thereto and the mixture was made uniform by stirring with a homo-mixer at 5000 rpm for 5 minutes. Aqueous solution of arginine as following preparation C was added thereto while further stirring. The resulting mixture was cooled to 40° C. or less while paddle-stirring, and was left standing.

preparation A:

| | |
|---|---|
| Squalane | 12% |
| Glycerol | 4% |
| Jojoba oil | 4% |
| Sodium surfactin | 1% |

preparation B:

| | |
|---|---|
| Stearic acid | 4% |
| Cetanol | 2% |
| Behenyl alcohol | 2% |
| Paraffin(56-58° C.) | 3% |
| p-aminobenzoic acid | 0.05% |

preparation C:

| | |
|---|---|
| L-Arginine | 0.1% |
| Water | bal. |

The obtained cream provided an excellent moisture retaining property and usability for feeling the smooth skin without skin irritation.

### EXAMPLE 23

### Nonaqueous Oily Thickened Gel-Like Composition

In accordance with the following preparation, sodium surfactin was dissolved in glycerol and thereto, squalane in small portions each was added while stirring. Additionally, clove oil was slowly added thereto while stirring.

| | |
|---|---|
| Squalane | 78% |
| Glycerol | 20% |
| Clove oil | 1% |
| Sodium surfactin | 1% |

The obtained nonaqueous oily thickened gel-like composition had a translucent appearance and when a cleansing cosmetic composition was prepared using this, unlike squalane, liquid dripping did not occur and an excellent cleansing property was exhibited. When a moisture retaining cosmetic composition was prepared using this, good attachment to skin and usability for feeling the smooth skin were provided without skin irritation.

### EXAMPLE 24

### Transparent Oily Thickened Gel-Like Composition

In accordance with the following preparation, sodium surfactin was dissolved in glycerol and thereto, squalane in small portions each was added while stirring. Additionally, water and orange oil were slowly added thereto while stirring.

| | |
|---|---|
| Squalane | 59% |
| Glycerol | 33% |
| Water | 6% |
| Orange oil | 1% |
| Sodium surfactin | 1% |

The obtained oily thickened gel-like composition had a transparent appearance, low viscosity and an excellent usability. When a cleansing cosmetic composition was prepared using this, unlike squalane, liquid dripping did not occur and an excellent cleansing property was exhibited. When a moisture retaining cosmetic composition was prepared using this, good attachment to skin and usability for feeling the smooth skin were provided without skin irritation.

### EXAMPLE 25

### Transparent Oily Thickened Gel-Like Composition

In accordance with the following preparation, sodium surfactin was dissolved in glycerol and thereto, squalane in small portions each was added while stirring. Additionally, water and oil of bitter orange were slowly added thereto while stirring.

| | |
|---|---|
| Squalane | 59% |
| Glycerol | 33% |
| Water | 6% |
| Oil of bitter orange | 1% |
| Sodium surfactin | 1% |

The obtained oily thickened gel-like composition had a transparent appearance, low viscosity and an excellent usability. When a cleansing cosmetic composition was prepared using this, unlike squalane, liquid dripping did not occur and an excellent cleansing property was exhibited. When a moisture retaining cosmetic composition was prepared using this, good attachment to skin and usability for feeling the smooth skin were provided without skin irritation.

### EXAMPLE 26

### Nonaqueous Oily Thickened Gel-Like Composition

In accordance with the following preparation, sodium surfactin was dissolved in glycerol and thereto, γ-oryzanol was added to dissolve while stirring, then olive oil in small portions each was added while stirring.

| | |
|---|---|
| Olive oil | 78% |
| Glycerol | 20% |
| γ-oryzanol | 1% |
| Sodium surfactin | 1% |

The obtained nonaqueous oily thickened gel-like composition had a transparent appearance, when a cleansing cosmetic composition was prepared using this, unlike olive oil, liquid dripping did not occur and an excellent cleansing property was exhibited. When a moisture retaining cosmetic composition was prepared using this, good attachment to skin and usability for feeling the smooth skin were provided without skin irritation.

### EXAMPLE 27

### Ultraviolet Absorbent Gel

In accordance with the following preparation, sodium surfactin was dissolved in glycerol. 4-t-butyl-4′-methoxybenzoylmethane was added to 2-ethylhexyl p-methoxycinnamate and heated to 80° C. to dissolve and was added in small portions to the sodium surfactin solution while stirring.

| | |
|---|---|
| 2-ethylhexyl p-methoxycinnamate | 68% |
| 4-t-butyl-4′-methoxybenzoylmethane | 11% |
| Glycerol | 20% |
| Sodium surfactin | 1% |

The obtained ultraviolet absorbent gel had a white appearance, and when this was blended as the substrate for a sun-screen cosmetics composition and the like, an extremely fine and stable emulsion were obtained.

### EXAMPLE 28

### Ultraviolet Absorbent Gel

In accordance with the following preparation, sodium surfactin was dissolved in glycerol. 4-t-butyl-4′-methoxybenzoylmethane was added to 2-ethylhexyl p-methoxycinnamate and heated to 80° C. to dissolve and was added in small portions to the sodium surfactin solution while stirring. Additionally, methylpolysiloxane and cyclomethicone in small portions each were added thereto while stirring.

| | |
|---|---|
| 2-ethylhexyl p-methoxycinnamate | 33% |
| 4-t-butyl-4′-methoxybenzoylmethane | 11% |
| Methylpolysiloxane | 21% |
| Cyclomethicone | 20% |
| Glycerol | 14% |
| Sodium surfactin | 1% |

The obtained ultraviolet absorbent gel had a white appearance, and when this was blended as the substrate for a sun-screen cosmetics composition and the like, an extremely fine and stable emulsion were obtained.

### EXAMPLE 29

### Ultraviolet Absorbent Emulsion

In accordance with the following preparation, sodium surfactin was dissolved in glycerol. 4-t-butyl-4′-methoxybenzoylmethane was added to 2-ethylhexyl p-methoxycinnamate and heated to 80° C. to dissolve and was added in small portions to the sodium surfactin solution while stirring. Then, methylpolysiloxane and cyclomethicone in small portions each were added thereto while stirring. Additionally, water was slowly added thereto while stirring.

| | |
|---|---|
| 2-ethylhexyl p-methoxycinnamate | 7.5% |
| 4-t-butyl-4′-methoxybenzoylmethane | 2.5% |
| Methylpolysiloxane | 5% |
| Cyclomethicone | 4% |
| Glycerol | 3% |
| Sodium surfactin | 1% |
| Water | 77% |

The obtained ultraviolet absorbent emulsion had a white appearance, and was an extremely fine and stable emulsion. This can be blended in a sun-screen cosmetics composition and the like.

### EXAMPLE 30

### Ultraviolet Absorbent Emulsion

In accordance with the following preparation, sodium surfactin was dissolved in glycerol. 4-t-butyl-4′-methoxybenzoylmethane was added to 2-ethylhexyl p-methoxycinnamate and heated to 80° C. to dissolve and was added in small portions to the sodium surfactin solution while stirring. Additionally, water was slowly added thereto while stirring.

| | |
|---|---|
| 2-ethylhexyl p-methoxycinnamate | 42% |
| 4-t-butyl-4′-methoxybenzoylmethane | 7% |
| Glycerol | 12.5% |
| Sodium surfactin | 1% |
| Water | 37.5% |

The obtained ultraviolet absorbent emulsion had a white appearance, and was an extremely fine and stable emulsion. This can be blended in a sun-screen cosmetics composition and the like.

### EXAMPLE 31

### Ultraviolet Absorbent Transparent Gel

In accordance with the following preparation, sodium surfactin and 70% aqueous sorbitol solution were dissolved in glycerol. 4-t-butyl-4′-methoxybenzoylmethane was added to 2-ethylhexyl p-methoxycinnamate and heated to 80° C. to dissolve, additionally, squalane was added and the mixture was made uniform, was added in small portions to the sodium surfactin solution while stirring.

| | |
|---|---|
| 2-ethylhexyl p-methoxycinnamate | 7.5% |
| 4-t-butyl-4′-methoxybenzoylmethane | 2.5% |
| Squalane | 57% |
| Glycerol | 16% |
| 70% aqueous sorbitol solution | 16% |
| Sodium surfactin | 1% |

The obtained ultraviolet absorbent gel had a transparent appearance, and can be used as a gel cosmetic composition for sun-screen. When this was blended as the substrate for a cream or a lotion for sun-screen cosmetic composition, an extremely fine and stable emulsion were obtained.

### EXAMPLE 32

### Nonaqueous Oily Thickened Gel-Like Composition

In accordance with the following preparation, sodium surfactin were dissolved in glycerol and thereto was added in small portions the mixture of the component the other than glycerol and sodium surfactin while stirring.

| | |
|---|---|
| Squalane | 10% |
| Sunflower seed oil | 10% |
| Glycerol tri(2-ethylhexanoate) | 10% |
| Jojoba oil | 10% |
| Isopropyl myristate | 10% |
| 2-Octyldodecyl myristate | 10% |
| Cetyl 2-ethylhexanoate | 10% |
| Methylpolysiloxane | 10% |
| Glycerol | 19% |
| Sodium surfactin | 1% |

The obtained nonaqueous oily thickened gel-like composition had a milky appearance, and can be used as a moisture retaining cosmetic composition for skin. When this was blended as the substrate for a cream or a lotion, an extremely fine and stable emulsion was obtained.

### COMPARATIVE EXAMPLE 1

### Moisture Retaining Cosmetic Composition

The moisture retaining cosmetic composition consisting of 100% of squalane oil was an oil having a transparent appearance. Although good attachment to skin and usability for feeling the smooth skin were provided, liquid drips due to low viscosity might cause to stain clothes or floor. Thus, there was a problem in the use property.

### COMPARATIVE EXAMPLE 2

### Moisture Retaining Cosmetic Composition

The moisture retaining cosmetic composition consisting of 100% of jojoba oil was an oil having a transparent appearance. Although good attachment to skin and usability for feeling the refreshed skin were provided, liquid drips due to low viscosity and might cause to stain clothes or floor. Thus, there was a problem in the use property.

### COMPARATIVE EXAMPLE 3

### Cleansing Oil

The cleansing oil consisting of 90% of mineral oil and 10% of olive oil had a transparent appearance and exhibited excellent cleansing property by well solubilizing a cosmetic composition, however, liquid drips due to low viscosity and might cause to stain clothes or floor. Thus, there was a problem in the use property.

### INDUSTRIAL APPLICABILITY

According to the present invention, an oily thickened gel-like composition extremely low in the skin irritation, capable of containing an oily ingredient in a large amount and suitable for a cleansing cosmetic composition, a moisture retaining cosmetic composition or the like; a preparation method of an emulsified composition using the oily thickened gel-like composition; an emulsified composition using the oily thickened gel-like composition; and a cosmetic composition comprising the oily thickened gel-like composition or the emulsified composition, such as cleansing cosmetic composition, moisture retaining cosmetic composition, cream and lotion, can be provided.

1. An oily thickened gel-like composition comprising an anionic surfactant, water and/or a polyhydric alcohol, and an oily ingredient.

2. The oily thickened gel-like composition as claimed in claim 1, wherein the anionic surfactant has a lipopeptide structure.

3. The oily thickened gel-like composition as claimed in claim 1, wherein the anionic surfactant is contained in an amount of 0.01 to 45 mass % and water and/or the polyhydric alcohol is contained in an amount of 0.01 to 43 mass %.

4. The oily thickened gel-like composition as claimed in claim 1, wherein the oily ingredient is contained in an amount of 1 to 99.9 mass %.

5. The oily thickened gel-like composition as claimed in claim 2, wherein the anionic surfactant having a lipopeptide structure is a salt of surfactin and/or a salt of the homologue of surfactin.

6. The oily thickened gel-like composition as claimed in claim 5, wherein the surfactin is a compound represented by the formula (I) or a composition comprising two or more kinds of the compound represented by the formula (I): 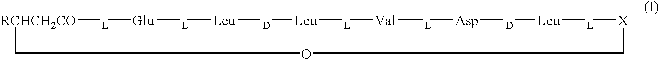
wherein X represents an amino-acid residue selected from the group consisting of leucine, isoleucine, valine, glycine, serine, alanine, threonine, asparagine, glutamine, aspartic acid, glutamic acid, lysine, arginine, cysteine, methionine, phenylalanine, thyrosin, tryptophan, histidine, proline, 4-hydroxyproline and homoserine, and R represents a normal alkyl group having from 8 to 14 carbon atoms, isoalkyl group having from 8 to 14 carbon atoms or anteisoalkyl group having from 8 to 14 carbon atoms.

7. The oily thickened gel-like composition as claimed in claim 6, wherein X in formula (I) is an amino-acid residue selected from leucine, isoleucine or valine.

8. The oily thickened gel-like composition as claimed in claim 5, wherein the salt is one or more salts selected from the group consisting of sodium salt, potassium salt, monoethanolamine salt, diethanolamine salt, triethanolamine salt, arginine salt and lysine salt.

9. The oily thickened gel-like composition as claimed in claim 5, wherein the salt of surfactin and/or the salt of the homologue of the is sodium surfactin.

10. The oily thickened gel-like composition as claimed in claim 1, wherein the polyhydric alcohol has three or more hydroxyl groups.

11. The oily thickened gel-like composition as claimed in claim 10, wherein the polyhydric alcohol having three or more hydroxyl groups is one or more alcohols selected from the group consisting of glycerol, diglycerol, polyglycerol, sorbitol, multitol, erythritol, pentaerythritol, glucose, saccharose and trehalose.

12. The oily thickened gel-like composition as claimed in claim 1, wherein the oily ingredient is a liquid oil.

13. The oily thickened gel-like composition as claimed in claim 12, wherein the liquid oil is one or more liquid oils selected from the group consisting of mineral oil, vegetable oil, hydrocarbon, ester oil, silicone oil and triglyceride.

14. The oily thickened gel-like composition as claimed in claim 12, wherein the liquid oil is one or more liquid oils selected from the group consisting of glycerol tri(2-ethylhexanoate), cetyl 2-ethylhexanoate, 2-octyldodecyl myristate, isopropyl myristate, liquid paraffin, squalane, jojoba oil, olive oil, rice embryo oil, sunflower seed oil and clove oil.

15. The oily thickened gel-like composition as claimed in claim 13, wherein the silicone oil is one or more liquid oils selected from the group consisting of dimethyl silicone oil such as octamethyltrisiloxane, decamethyltetrasiloxane, methyl-polysiloxane and high-polymerized methylpolysiloxane, cyclic silicone oil such as octamethylcyclotetrasiloxane, decamethylcyclopentasiloxane, dodecamethylcyclohexasiloxane, methylcyclopolysiloxane and methylpolycyclosiloxane, poly ether modified silicone oil, methylphenyl silicone oil such as methylphenylpolysiloxane.

16. The oily thickened gel-like composition as claimed in claim 12, wherein the liquid oil comprises a liquid oil having a property of ultraviolet absorption.

17. The oily thickened gel-like composition as claimed in claim 16, wherein the liquid oil having a property of ultraviolet absorption is 2-ethylhexyl p-methoxycinnamate or 2-ethylhexyl p-methoxycinnamate solution of 4-t-butyl-4′-methoxybenzoyl-methane.

18. The oily thickened gel-like composition as claimed in claim 1, which is transparent.

19. The oily thickened gel-like composition as claimed in claim 18, wherein a difference of refractive index n_{D}²⁰ between oil phase and aqueous phase in the oily thickened gel-like composition is 0.01 or less.

20. A method for preparing an emulsified composition, comprising adding water to the oily thickened gel-like composition described in any one of claims 1 to 19 to emulsify the composition.

21. An emulsified composition obtained by the preparation method described in claim 20.

22. A cosmetic composition containing the emulsified composition described in claim 21.

23. A cosmetic composition containing the oily thickened gel-like composition described in any one of claims 1 to 19.
